(19) United States
(12) Patent Application Publication
(10) Pub. No.: US 2025/0243545 A1
(43) Pub. Date: Jul. 31, 2025

(54) IN SITU HYBRIDIZATION METHODS FOR USE WITH FROZEN BONE MARROW ASPIRATE

(71) Applicant: Leica Biosystems Richmond, Inc., Richmond, IL (US)

(72) Inventors: Tatiana Johnston, Barrington, RI (US); Ina Menzl, Tucson, AZ (US); Nnamdi Okeke, Amesbury, MA (US)

(21) Appl. No.: 19/038,263

(22) Filed: Jan. 27, 2025

Related U.S. Application Data
(60) Provisional application No. 63/627,463, filed on Jan. 31, 2024.

(51) Int. Cl. C12Q 1/6883; C12Q 1/37; C12Q 1/6841; G01N 1/30; G01N 1/31; G01N 35/00; G01N 35/10

(52) U.S. Cl. CPC C12Q 1/6883; C12Q 1/37; C12Q 1/6841; G01N 1/31; G01N 35/0099; G01N 35/1002; G01N 35/1009; G01N 2001/305

(57) ABSTRACT

A method and apparatus for preparing a bone marrow aspirate (BMA) sample for detection of a targeted nucleic acid sequence including a first fixation step including contacting said BMA sample with a first fixation solution including formalin, and a second fixation step including contacting said BMA sample with a second fixation solution including methanol and acetic acid.

### CROSS REFERENCE TO RELATED APPLICATIONS

This application claims priority to and benefit of U.S. Provisional Application Ser. No. 63/627,463, filed Jan. 31, 2024, the contents of which are incorporated by reference in their entirety for all purposes.

### BACKGROUND

Bone marrow aspiration and biopsies are often performed both in the inpatient and outpatient settings to aid in the diagnosis and treatment of various blood disorders and diseases. Bone marrow aspiration and biopsy may be used, for example, for definitive diagnosis of hematologic disorders such as leukemia, multiple myeloma, lymphoma, unexplained anemia, and myelodysplastic syndrome. Retrieval of bone marrow, however, is an invasive and painful procedure, putting the patient at risk for post-operative bleeding, hemorrhage, cardiac tamponade, infection, and death, such that it is of great benefit to use frozen samples and avoid repeated aspiration.

Examination of the bone marrow (BM) aspirates, in many instances, is essential for the diagnosis of BM and blood disorders, which are commonly associated with gross chromosomal abnormalities. Reliable tests for the diagnosis of chromosomal abnormalities allow clinicians to decide between standard-of-care therapy and personalizing the treatment plan. One of the most run genetic tests in clinics is the Fluorescent in situ hybridization (FISH) assay which utilizes sequence-specific fluorescent-labeled probes to detect chromosomal rearrangements, which are visualized under a fluorescent microscope. The method may be used for example, for detection of chromosomal rearrangements, diagnosis, risk stratification, detection of minimal residual disease, and assessment of response to therapy. FISH is a useful tool for studying neoplastic disorders involving various chromosomal abnormalities. However, there is a need for improved methods that allow for FISH assays to be used with a bone marrow aspirate smear on a slide, particularly BMA that has been previously frozen without cryopreservative, as the cells in frozen BMA samples are fragile for post-thawing manipulation.

As such, the instant disclosure seeks to address one or more of the aforementioned needs in the art.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an example of a slide-staining instrument;

FIG. 2 depicts a perspective view of a tray for use with the slid-staining instrument of FIG. 1;

FIG. 3 depicts another perspective view of the tray of FIG. 2 with a slide and slide cover loaded onto the tray;

FIG. 4 depicts a perspective view of a slide staining assembly of the slide-staining instrument of FIG. 1;

FIG. 5 depicts a flow chart of an example of a sample preparation for use with the slide-staining instrument of FIG. 1;

FIG. 6 depicts a flow chart of an example of a FISH protocol for use in the slide preparation protocol of FIG. 5 and the slide-staining instrument of FIG. 1; and

FIG. 7 depicts a schematic diagram of a control system configured to implement the FISH protocol of FIG. 5 using the slide-staining instrument of FIG. 1.

FIG. 8 depicts the results of a FISH performed using Carnoy's fixation method on frozen BMA smears. No KMTA probe hybridization was observed (i.e., absence of fluorescence signal). Images are from AML BMA frozen sample. The absence of fluorescence of KMT2A probe signals indicates that conditions are not favorable for the FISH assay.

FIG. 9 depicts a comparison between formalin-fixed+Carnoy's treated slide (left) and Carnoy's only (right) slide. Formalin fixed frozen BMA showed distinctive FISH probes fluorescence signals (left, arrow) which were absent in Carnoy's only fixed BMA sample, right.

FIG. 10 depicts KMT2A gene rearrangements in clinical AML BMA samples. The enlarged image shows expected KMT2A gene rearrangement 1 Fusion (1F), 1 Red (1R), 1 Green (1G). This figure illustrates that the KMTA2A gene rearrangement can be visualized. Separate Red and green signals indicate a rearrangement, whereas yellow (Red+Green) indicates no rearrangement.

FIG. 11A-11C depicts FISH results for 3 hours formalin treatment, air dried (11A), 3 hours formalin treatment, BOND-III wash (11B), 3 hours formalin, PBS wash (11C). The 3 hours formalin, PBS wash resulted in the best FISH fluorescent signals.

FIGS. 12A and 12B depict FISH results for 3 hours formalin+PBS wash (12A) and FISH results using the two-step fixation method comprising formalin followed by Carnoy's solution (12B).

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

### Example Slide-Staining Instrument

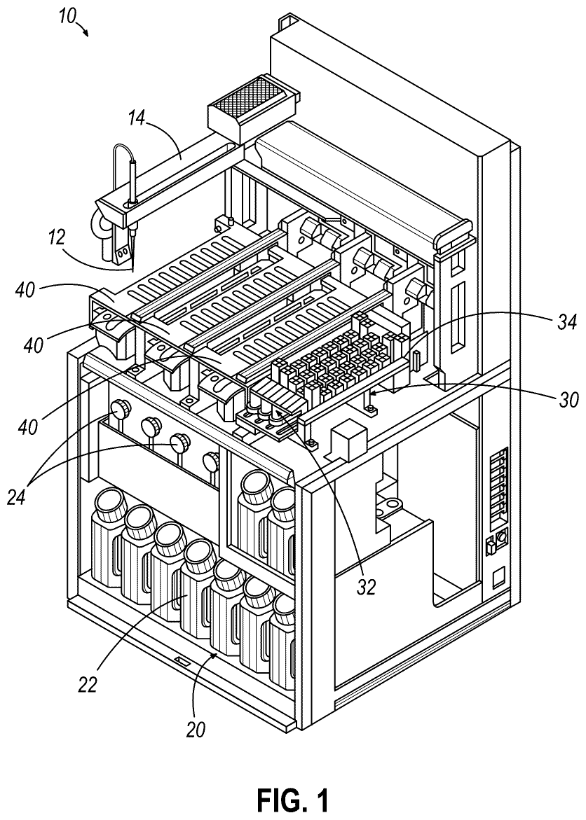

In one aspect, disclosed are novel methods for the preparation of samples for fluorescent in situ hybridization (FISH) detection methods which may employ, in whole or in part, the slide- staining instrument (10) described herein. FIG. 1 shows an example of a slide-staining instrument (10) that is generally configured to automatically stain tissue mounted on microscope slides. Slide- staining instrument (10) includes a group fluid dispenser (12) (also referred to as an aspirating probe) mounted to a robotic arm (14) and a plurality of slide staining assemblies (40) disposed beneath robotic arm (14). As will be described in greater detail below, robotic arm (14) and slide staining assemblies (40) are generally configured to operate cooperatively to automatically dispense reagents, for example, FISH assay reagents, stain, probes, enzymes, and the like, onto one or more microscope slides disposed within each slide staining assembly (40).

To supply reagents, slide-staining instrument (10) includes a bulk container rack (20) and a reagent platform (30). Bulk container rack (20) is configured to hold a plurality of bulk containers (22). Bulk containers (22) may be configured to contain a relatively large quantity of reagent that may be communicated to group fluid dispenser (12) and/or each slide staining assembly (40). Examples of reagents that may be stored in bulk containers (22) may include, for example, formalin solutions, wash solutions, deionized water, buffered solutions such as phosphate buffered saline (PBS), and/or alcohol, and additional reagents useful to carrying out the disclosed methods. Additionally, one or more of bulk containers (22) may be configured to contain waste fluids communicated from group fluid dispenser (12) and/or each slide staining assembly (40). In some examples, such waste fluids may be separated by slide-staining instrument (10) into bulk waste and hazardous waste. Thus, separate bulk containers (22) may be included for bulk waste and hazardous waste. A plurality of syringe pumps (24) may be included proximate bulk containers (22) to communicate fluid to or from bulk containers (22) relative to other portions of slide-staining instrument (10).

Reagent platform (30) is generally configured to receive one or more reagent trays (32). Each reagent tray (32) is configured to hold a plurality of reagent containers (34). Reagent containers (34) may be configured to contain a relatively small quantity of reagent for communication to group fluid dispenser (12) and/or slide staining assembly (40). Each reagent tray (32) may include a particular predetermined combination of reagent containers (34) to form a predefined reagent system. Such predefined reagent systems may be ready-to-use and are discarded upon exhaustion during use with a particular protocol. Each reagent tray (32) is registered upon insertion into reagent platform (30). Although not shown, it should be understood that group fluid dispenser (12) may access reagent containers (34) directly. Also, one or more syringe pumps similar to syringe pumps (24) described above may be included to communicate reagents from reagent containers (34) to group fluid dispenser (12), slide staining assembly (40), and/or other portions of slide-staining instrument (10).

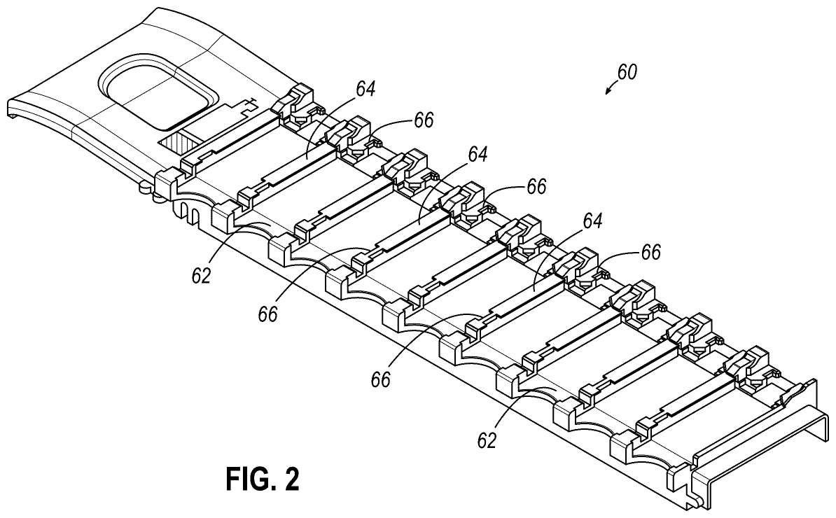

FIG. 2 shows an example of a slide tray (60) that may be used with slide-staining instrument (10) to hold one or more slides during the application of reagents via slide-staining instrument (10). Slide tray (60) includes a plurality of slide recesses (62) separated by rails (64). Each slide recess (62) is generally configured to receive an individual slide. Although not shown, it should be understood that each slide recess (62) may include an open bottom in some examples such that each slide may be supported within slide tray (60) at the periphery of each slide rather than in a center or middle of each slide. Thus, in some examples, each slide recess (62) may include one or more ledges, protrusions, supports or other features configured to support the outer edges of a slide received therein.

Slide tray (60) further includes a plurality of couplers (66) with one or more couplers (66) being associated with a corresponding slide recess (62). Couplers (66) are generally configured to removably couple to a cover (70) (also referred to as a covertile). As will be described in greater detail below, covers (70) may be used to secure a slide in position on or within each slide recess (62). In some examples, couplers (66) may include one or more recesses on one side of a counterpart slide recess (62) and one or more snap-fits on another side of a counterpart slide recess (62). Of course, in other examples, couplers (66) may have a variety of other configurations as will be apparent to those of ordinary skill in the art in view of the teachings herein.

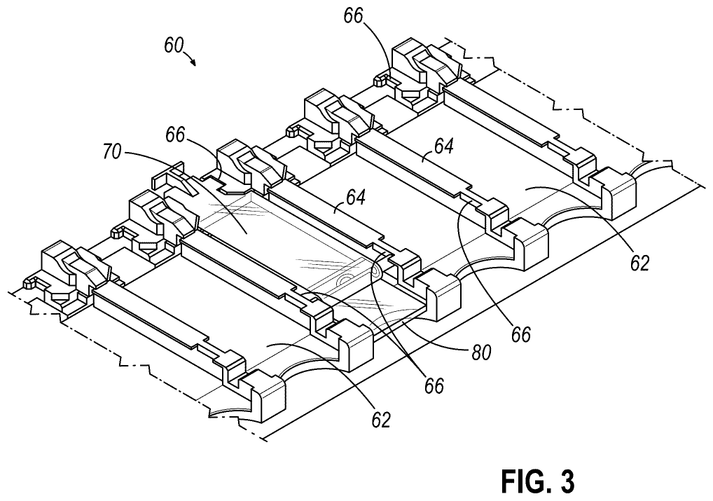

FIG. 3 shows the assembly of slide tray (60) and cover (70) to hold a slide (80) in greater detail. As can be seen, slide (80) may be disposed between cover (70) and slide tray (60) within a given slide recess (62). Cover (70) is generally configured to hold slide (80) in position and rest over slide (80) during staining. In this configuration, capillary action may draw reagent dispensed to slide (80) between cover (70) and slide (80). Such capillary action may be desirable to provide gentle and uniform tissue coverage with the dispensed reagent, thereby minimizing the volume of reagent used and protecting tissue from drying between applications. In some examples, cover (70) may be reusable, while cover (70) may be disposable in other examples.

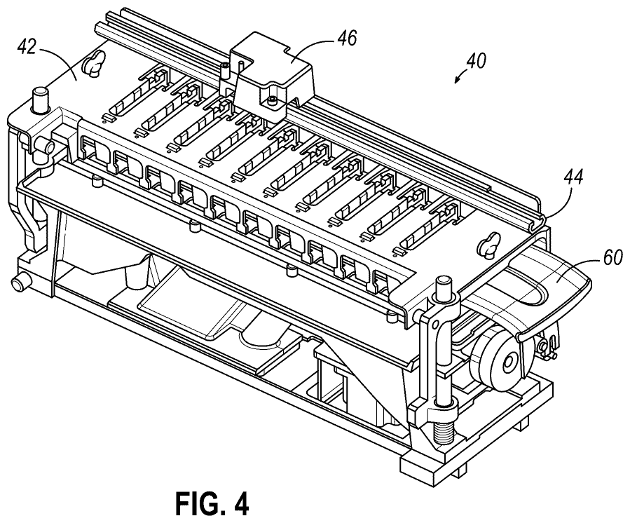

FIG. 4 shows an example of slide staining assembly (40) in greater detail. As described above, slide-staining instrument includes a plurality of slide staining assemblies (40) generally configured to operate cooperatively with robotic arm (14) to automatically dispense reagents onto one or more microscope slides disposed within each slide staining assembly (40). Specifically, each slide staining assembly (40) is configured to receive a corresponding slide tray (60). When slide tray (60) is loaded with slides such as slide (80) robotic arm (14) along with slide staining assembly (40) itself may be used to dispense reagents onto each slide loaded on slide tray (60).

Slide staining assembly (40) includes a top cover (42), a guide rail (44), and a batch fluid dispenser (46). Slide staining assembly (40) is configured removably receive and support slide tray (60) beneath top cover (42). Batch fluid dispenser (46) is configured to move along guide rail (44) relative to slide tray (60) to dispense slide reagents and buffers from one or more of bulk containers (22) onto slides contained within slide tray (60). In some examples, batch fluid dispenser may alternatively be characterized as a fluid dispensing robot. Although not shown, it should be understood that slide staining assembly (40) may include a plurality of heating elements corresponding to each slide recess (62) of slide tray (60). A portion of such heating elements can be incorporated into a support for each slide, which can be configured to communicate heat from heating elements to each slide. Thus, slide staining assembly (40) is configured to maintain each slide disposed within slide tray (60) at a selected temperature independently of the other slides.

In use, robotic arm (14) is used to position group fluid dispenser (12) relative to slide staining assembly (40) and reagent platform (30). Robotic arm (14) may then be moved to reagent platform (30) to gather one or more reagents with group fluid dispenser (12). Optionally, one or more reagents may be mixed or otherwise manipulated in a separate area of slide-staining instrument (10). Robotic arm (14) may then move to a selected slide staining assembly (40) to dispense gathered reagents onto one or more slides disposed within the selected slide staining assembly (40).

Simultaneously with use of robotic arm (14) to dispense reagents, or separately therefrom, each slide staining assembly (40) may dispense reagents onto one or more slides disposed within a respective slide staining assembly (40). Specifically, batch fluid dispenser (46) may move along guide rail (44) to communicate one or more reagents from bulk containers (22) onto slides via syringe pumps (24) and other components associated with slide-staining instrument (10). Thus, robotic arm (14) and slide staining assemblies (40) are configured to operate cooperatively to efficiently complete staining protocols with one or more steps of a given staining protocol being performed simultaneously with one or more other steps. During the performance of such staining protocols, heaters included within each slide staining assembly (40) are used to maintain the temperature of individual slides at a selected temperature in accordance with such staining protocols.

### Sample Preparation Protocol (100)

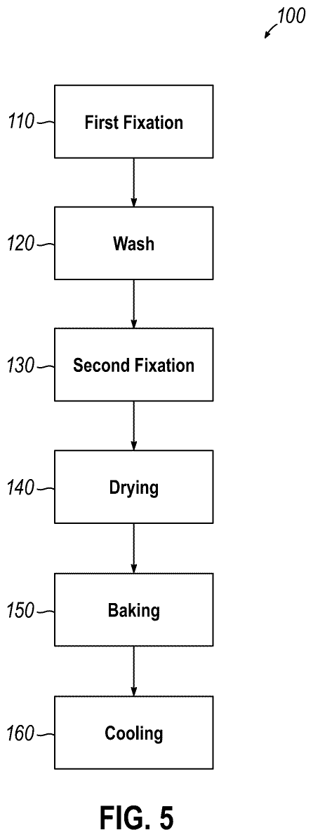

FIG. 5 depicts the sample preparation protocol (100), which may be used on, for example, non-processed BMA samples, which have been previously frozen. A “non-processed BMA sample” encompasses a BMA sample that has not been centrifuged and separated into a liquid and solid component; rather, the non-processed BMA samples generally contain the entirety of the sample as obtained from the patient. In general, the non-processed BMA sample contemplated herein is a previously frozen BMA sample.

Prior to the sample preparation protocol (100), a freshly thawed BMA sample is applied to a slide via a “squash” method, in which about 5 μL to about 100 μL, or about 10 μL to about 50 μL, or about 15 μL to about 25 μL, or about 20 μL of previously frozen BMA is applied to a slide. Another glass slide with an uncharged surface is then contacted, edge first, to the slide with the BMA sample, and lowered on the BMA sample. The slide is then gently pulled over the sample with minimal pressure to spread the BMA sample, thus creating a “smear.” The BMA smear can then be used in the sample preparation protocol (100).

Sample preparation protocol (100) begins with a first fixation step (110). First fixation step (110) generally comprises contacting a BMA sample with a 10% formalin solution, though it should be understood that a range of formalin may be used. The first fixation step (110) is followed by wash step (120), which may be carried out for a predetermined time at a predetermined temperature, and which may be repeated 1-3 times. Wash step (120) may be performed to remove any remaining formalin solution. In the present example, wash step (120) is performed using a PBS buffer solution, pH 7.4 for 5 minutes. In other examples, different buffer solution may be used. In yet other examples, wash step (120) may be performed using other solutions such as deionized water. Wash step (120) of the present example may include submerging the slide in a volume of wash buffer at ambient temperature to completely cover the sample on a slide. The wash step (120) may be repeated one to five times, replacing the wash solution between wash steps. In other examples, a variety of alternative dispenses may be used such as more than five, or less than five.

Following wash step (120), a second fixation step (130) is carried out, which typically employs contact and incubation of the sample with a Carnoy's solution as described herein (methanol and acetic acid at a 3:1 ratio) for a period of from about 1 minute to about 20 minutes, or from about 5 minutes to about 15 minutes or about 10 minutes. Following second fixation step (130), drying step (140) is carried out. Drying step (140) may be carried out at room temperature until dry, for example for a period of time from about 15 to 20 minutes, or until fully dry by visual examination. Baking step (150) is then carried out in which the slides are baked at 80° C. for 10 min. The slides are then subjected to a cooling step (160), in which the slides are brought to room temperature. Slides may then be labeled, cover tiled, and placed on a tray for an automated FISH assay carried out on slide-staining instrument (10). In other examples, various alternative time and/or temperature parameters may be used as will be apparent to those of ordinary skill in the art in view of the teachings herein.

FIG. 5 depicts an exemplary sample preparation protocol (100). Such protocol may be performed outside of the slide-staining instrument (10).

### FISH Assay (200)

As described above, slide-staining instrument (10) may be configured to perform a variety of protocols where robotic arm (14) and/or slide staining assemblies (40) may automatically apply a variety of reagents to slides under various predetermined parameters. Such slide staining protocols may include a series of steps where various reagents may be applied to individual slides in a predetermined order, in a predetermined volume at predetermined temperatures for predetermined periods of time.

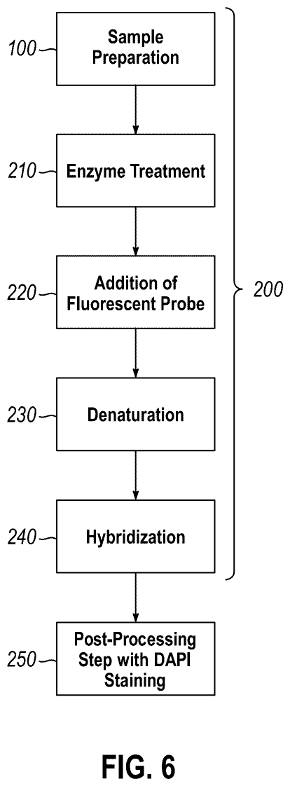

FIG. 6 depicts the steps of the FISH assay (200) as carried out on a BMA sample prepared according to sample preparation protocol (100). Following sample preparation protocol (100), an enzyme treatment (210) is carried out. For example, the enzyme as provided in the BOND Enzyme Pretreatment Kit may be used. The BOND Enzyme Pretreatment Kit comprises an enzyme concentrate and an enzyme diluent, which is then mixed and used for enzymatic digestion of sectioned formalin-fixed tissues. The enzyme treatment on formalin-fixed tissue exposes epitopes that have been masked by formalin fixation, allowing accessibility of the primary antibody to the epitope. BOND Enzyme Concentrate (1 mL) contains a proteolytic enzyme (17 mg/mL) and a stabilizer. The enzyme treatment step may be carried out at a temperature sufficient to maintain enzymatic activity and may be carried out for a period of time ranging from about 30 seconds to about 5 minutes, or from about 1 minute to about 10 minutes, or about 2 minutes. The duration and temperature used for incubation of the enzyme during the enzyme treatment (210) may be predetermined and programmed into the slide-staining instrument (10) and may depend on the specific enzyme used.

Following enzyme treatment (210), a probe addition step (220) is carried out, in which a probe specific for a predetermined nucleic acid sequence is contacted with the prepared sample. The selection of the probe is determined based on the target gene of interest and is designed to provide information as to the presence or absence of a gene rearrangement. Various suitable probes for use with step (220) will be apparent to those of ordinary skill in the art in view of the teachings herein. In general, the probe will be a nucleic acid probe conjugated to a detectable label. In a further aspect, the detectable label is a fluorescent label. In general, the nucleic acid probes may be at least two different probes and may comprise distinct detectable labels, for example at least two different colors. Exemplary FISH methods for detecting gene rearrangements are described in, for example, Wang, L., Basturk, O., Wang, J. et al. A FISH assay efficiently screens for BRAF gene rearrangements in pancreatic acinar-type neoplasms. Mod Pathol 31, 132-140 (2018). https://doi.org/10.1038/modpathol.2017.106

Following the probe addition step (220), the sample is subjected to denaturation (230), followed by an incubation that allows for hybridization of labeled probes to the targeted complementary sequence, shown in FIG. 6 as the hybridization step (240). The predetermined time and temperature of the hybridization step (240) depends on the specific probe used. In one aspect the hybridization is for about 12 hours, or from about 10 hours to about 15 hours, or from about 7 hours to about 20 hours, depending on the specific nucleic acid probe composition and length and hybridization buffer.

Following hybridization step (240), a post-processing step with DAPI staining (250) may be carried out. DAPI (4,6-diamidino-2-phenylindole) is used as a counterstain in combination with fluorescence labeled FISH probes to enhance visualization of the chromosomal rearrangements. The primary role of DAPI in this context is to stain nuclear double stranded DNA. Following post-processing, the slide may be visualized. In one aspect, a microscope equipped with a camera and appropriate software is used to capture and analyze fluorescence signals from the FISH probes to detect possible gene rearrangements in the BMA sample.

It should be understood that the above protocols may include one or more washing steps between any one or more of the steps or protocols described above prior to the dehydration step with increased concentration of alcohol ranging from 70% to 100%. Such wash steps may be performed with a variety of solutions such as deionized water, phosphate buffered saline, or other buffered solutions, or various combinations thereof.

Incubation times of any of the described steps may vary. For example, any dispense cycle in which a reagent is applied to the sample may include an incubation. Incubations are generally performed with the given slide being heated to a predetermined temperature. The incubation time may be varied depending on the desired outcome and the particular reagents (for example, the particular probe) that is used. For example, the incubation time may be about 20 minutes for a dispense cycle, or about 30 minutes for a dispense cycle, or about 60 minutes for a dispense cycle. Although particular incubation times are described, it should be understood that in other examples, either a different minimum incubation time or a different maximum incubation time may be used.

### Diagnosis and Treatment

The methods described herein may further be used for the diagnosis and treatment of a disease. For example, in one aspect, the disclosed methods may be used for the diagnosis and/or treatment of a leukemia. In this aspect, a sample is obtained from an individual having, or suspected of having, leukemia. The sample is then subjected to the methods disclosed herein. For example, the sample may be an unprocessed, previously frozen BMA sample. The sample may be subjected to the sample preparation protocol (100), followed by the FISH assay (200). Following post-processing and visualization of the processed sample, an individual may be diagnosed. Based on the diagnosis, an appropriate therapy may be determined.

### Control System (310)

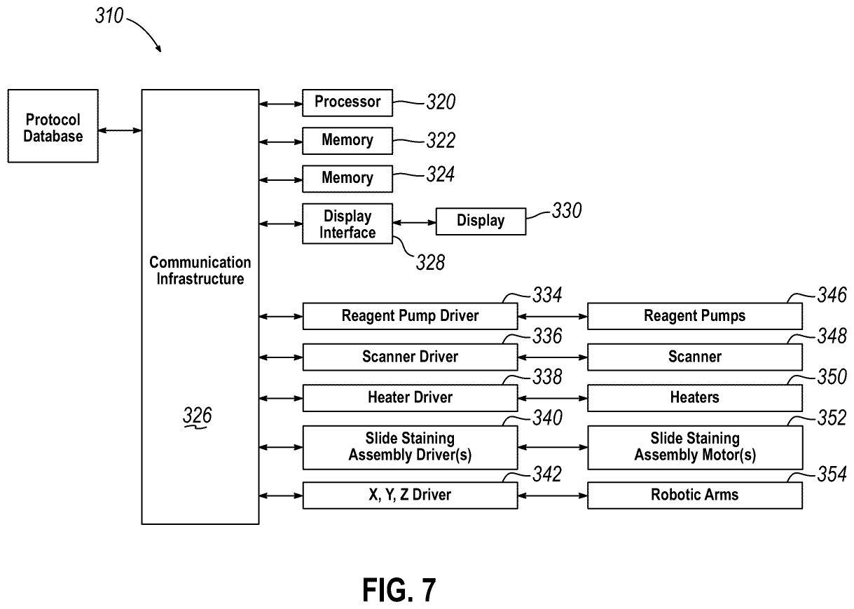

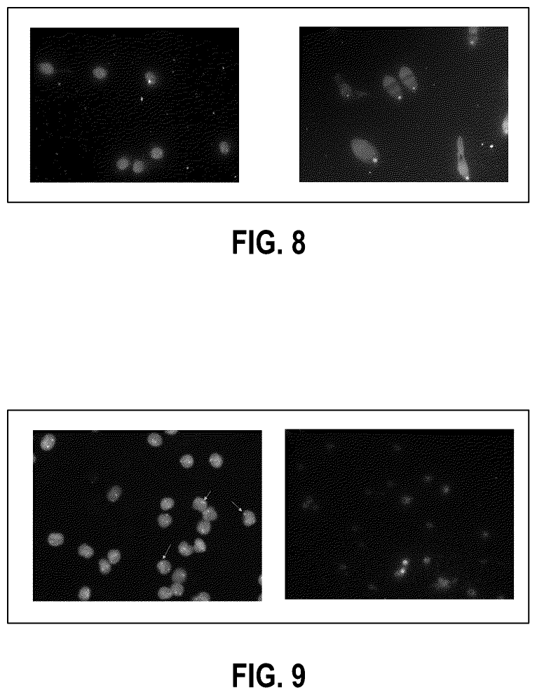

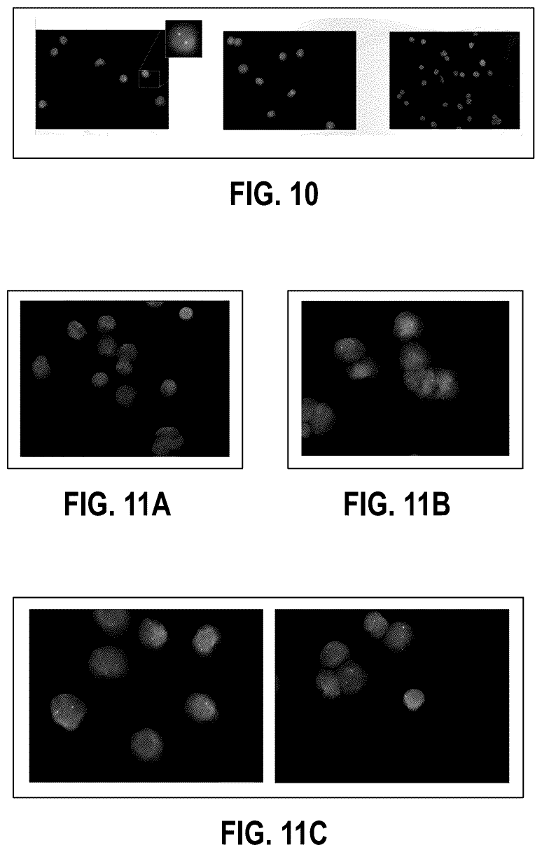

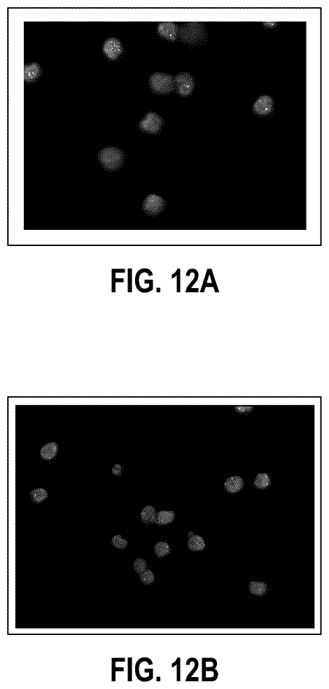

FIG. 7 shows an example of a control system (310) that may be used to implement the FISH assay (200) as described herein using slide-staining instrument (10). Control system (310) includes a processor (320) in communication with a first memory device (322) for storing computer program code and a second memory device (324) for storing data generated by processor (320) when implementing the computer program code, via communications infrastructure (326). Optionally, the functions of first memory device (322) and second memory device (324) may be combined in a single memory device or a plurality of memory devices in communication with each other.

Control system (310) further includes one or more inputs and one or more outputs to facilitate operator interaction with control system (310). For instance, in the present example, control system (310) includes a display interface (328) and corresponding display (330) to enable operator interaction with control system (310). In some examples, display (330) may be configured as a touch screen display so display (330) may both receive operator input and communicate operator outputs. In addition, or in the alternative, control system (310) may include one or more operator input features such as a keyboard or keypad to provide a dedicated operator input feature.

Control system (310) also includes driver modules (334, 336, 338, 340, 342) for controlling the motors, pumps, scanners, heaters and other devices (346, 348, 350, 352, 354) used for operation of the slide-staining instrument (10). It will be appreciated that in FIG. 7, examples only of driver modules and devices are depicted, and a person of skill in the art will be able to determine the driver modules and devices required to implement slide-staining instrument (10) to provide the functionality described herein.

It should be understood that memory devices (322, 324) may be disposed within a portion of slide-staining instrument (10) or may be hosted remote from slide-staining instrument (10) in data communication with processor (320). Regardless of the specific configuration, the processor (320) is configured to read instructions from first memory device (322) to operate the slide-staining instrument (10) to complete one or more slide-staining protocols. Such staining protocols (e.g. order of reagents to be dispensed by batch fluid dispenser (46) and group fluid dispenser (12) to slides) are stored in a protocol database (356) accessible by processor (320) via communications infrastructure (326), such that processor (320) can configure batch fluid dispenser (46) and group fluid dispenser (12) to dispense reagents to slides disposed within each slide staining assembly (40) in a predetermined order and at predetermined intervals.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

### Example I. Two-Step Preparation Protocol for BMA Samples for Fluorescent in Situ Hybridization (FISH) Assay

The following two-step fixation protocol is used for preparation of previously frozen BMA for a FISH assay. The method may be performed prior to introduction to automated processing on slide-staining instrument (10):
(a) A frozen BMA sample is thawed at 37° C. for approximately 2 minutes and gently mixed by inverting the vial before taking aliquots for placing on the slide.
(b) 20 μl of AML BMA sample is placed on the top of the slide; a second glass slide is used to “squash” the sample to make the smear.
(c) The slides are dried at room temperature for approximately one hour and examined for complete dryness before proceeding to the next step. This step is generally carried out in a BSL-2 hood.
(d) First Fixation: Dried slides are immersed in a 10% formalin solution prepared from Buffered Formalin Concentrate 5 (Leica Biosystems, #3800540) according to the manufacturer's instructions and incubated for 3 hours.
(e) After the fixation in formalin, the slides are washed three times in PBS, pH 7.4 for 5 min.
(f) Second Fixation: Washed slides are directly placed in freshly prepared Carnoy's solution (a mixture of methanol and acetic acid at a 3:1 ratio), for 10 min.
(g) After the Second Fixation step, the slides are left to dry under the chemical hood at room temperature for 15-20 min and examined for complete dryness before proceeding to the next step.
(h) Slides are baked at 80° C. for 10 min, cooled to room temperature, labeled, cover tiled, and placed on a tray for an automated FISH assay carried out on slide-staining instrument (10).
(i) Incubation steps may be performed in Coplin staining jars. The washes may be done in a regent vessel w/handle (Leica Biosystems, #140475336590) or Coplin staining jars.

Reagents used: Buffered Formalin Concentrate 5, Leica Biosystems, #3800540; Carnoy's solution (mixture of methanol and acetic acid at 3:1 ratio, prepared fresh just before use); Acetic acid, VWR, #0714-500ML; 100% methanol, Millipore Sigma, #MX0485-7; PBS, pH 7.4, ThermoFisher Scientific, #10010023.

The sample preparation steps outlined above are generally performed outside of slide-staining instrument (10).

### Example II. Detection of KMT2A Gene Rearrangements in BMA Samples Using FISH Assay

KMT2A gene rearrangements are responsible for 5-10% of cases of acute myeloid leukemia (AML). KMT2A is Lysine (K)-Specific Methyltransferase 2A, also known as MLL1 (Mixed Lineage Leukemia 1). Patients with rearrangements in the KMT2A gene have a poor overall survival (5-year OS of 20%) with a high rate of resistance and relapse following recommended standard of care treatment. Thus, early detection of KMT2A gene rearrangements is essential for personalized treatment of patients with this type of leukemia. The disclosed sample preparation protocols may be used on previously frozen, unprocessed BMA samples, such as from individuals diagnosed with AML, which may be assayed using an automated KMT2A FISH assay on the slide-staining instrument (10) described herein to detect a KMT2A gene rearrangement.

Unprocessed BMA is a non-standard sample type for a FISH assay, and to Applicant's knowledge, clinicians do not perform FISH tests on frozen BMA smears or on BMA smears in general. Rather, fresh bone marrow, acquired through an invasive procedure on a patient, undergoes a “harvest process” before isolated cells from BMA are placed on a slide and processed for cytogenetic analysis. In contrast, the described methods allow for successful FISH assay methods on unprocessed, previously frozen BMA smears. The present example is exemplary in nature; selection of appropriate hybridization probes for FISH assays for the detection of a gene of interest and/or a gene rearrangement will be understood by one of ordinary skill in the art.

The BMA sample preparation protocol is described above. In brief, a 20 μl drop of the BMA sample is smeared on a glass slide, airdried for 1 hour 20 minutes, fixed in 10% formalin buffered solution for 3 hours, rinsed in PBS 3×5 minutes at pH 7.4, placed in freshly prepared Carnoy's solution (methanol and acetic acid at a 3:1 ratio) for 10 minutes, airdried for 20 minutes, baked at 80° C. for 10 minutes, and allowed to cool before placing on the slide-staining instrument (10), which is programmed to carry out the desired FISH assay.

The FISH protocol run on the Leica BOND III with Enzyme 5 (proteinase, 1:3000 dilution) for 2 minutes, KMT2A gene probes (1:10 dilution), 95° C. denaturation for 10 minutes, 12 hours hybridization, and embedding the slides in antifade mounting medium with 0.1 μg/mL DAPI and cover slipped after the slides were post-processed. The Kreatech™ FISH probes manufactured by Leica Biosystems for detection of KMT2A gene rearrangements were used in defining conditions for KMT2A FISH assay on the BOND-III. The selected parameters allowed KMT2A gene probes to hybridize to the target sequence and KMT2A gene rearrangements can be visualized under fluorescent microscope and assessed by a trained pathologist. The protocol for KMT2A FISH assay on the BOND-III is as follows (“*” indicates a pre-defined protocol pre-programmed into the BOND-III):

| BOND Protocol Type | Protocol Name | Comments |
|---|---|---|
| Staining protocol | *FISH Protocol C | KMT2A probes at 1:10 |
| Preparation protocol | *Frozen Slide Delay | Schedule the instrument |
| | | finish time for |
| | | 12 h FISH run |
| Enzyme protocol | Enzyme 5 for 2 min | Enzyme 5 1:3000 |
| Denaturation | *Denaturation (10 min) | 2 min 95° C. 10 min |
| Hybridization | *ISH Hybridization | N/A |
| | (12 Hr) | |

Sample and Slide Preparation. BMA smear is prepared as in Example I and the BOND-III run conditions were as described in Table above in the BSL-2 cabinet.

After the BOND-III run, slides are post-processed as follows:
(a) Two-minute incubation in distilled water.
(b) Dehydration in ethanol series (70-95-100%, each 1 min).
(c) Air dry for 15-20 minutes at room temperature in chemical hood or until completely dry.
(d) Embed in 0.1 μg/ml DAPI diluted in antifade mounting medium and coverslip. (DAPI Counterstain (1μg/ml) in antifade diluent, Leica Biosystems, LK-096A.)
(e) Seal with a coverslip sealant, allow to dry, and view under the fluorescent microscope.

The DM5500B microscope equipped with 365FX camera and Cyto Vision software (RUO) is used to capture and analyze fluorescence signals from KMT2A gene FISH probes to detect possible gene rearrangements in frozen AML BMA samples and to evaluate different BOND-III protocol conditions used on AML BMA samples. All the images are taken on 63× oil objective either in Capture Screen or Probe Capture (allows scoring) modes.

### IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Exemplary Combinations

### Example 1

A method of processing a bone marrow aspirate (BMA) sample for detection of a targeted nucleic acid sequence comprising a first fixation step comprising contacting said BMA sample with a first fixation solution comprising formalin, and a second fixation step comprising contacting said BMA sample with a second fixation solution comprising methanol and acetic acid.

### Example 2

The method of example 1, said BMA being an unprocessed BMA sample.

### Example 3

The method of example 1 or 2, said BMA being previously frozen.

### Example 4

The method of any of examples 1 through 3, said first fixation solution comprising from about 5% to about 20% formalin.

### Example 5

The method of any of examples 1 through 4, said first fixation solution comprising 10% formalin.

### Example 6

The method of any of examples 1 through 5, said second fixation solution comprising methanol and acetic acid at a ratio of about 3 to about 1.

### Example 7

The method of any of examples 1 through 6, said second fixation step being carried out following said first fixation step.

### Example 8

The method of any of examples 1 through 7, said second fixation step being followed by a drying step.

### Example 9

The method of any of examples 1 through 8, said BMA sample being applied to a glass slide prior to said first step, placing a second slide at an angle to said first slide;
lowering said second slide to cover the BMA sample placed on said first slide; and
sliding said second slide across said first slide to create a BMA sample smear.

### Example 10

The method of any of examples 1 through 9, further comprising contacting said BMA sample with a proteolytic enzyme.

### Example 11

The method of any of examples 1 through 10, further comprising contacting said BMA sample with at least one labeled nucleic acid probe.

### Example 12

The method of example 11, said at least one labeled nucleic acid probe being fluorescently labeled.

### Example 13

The method of any of examples 11 through 12, said at least one labeled nucleic acid probe comprising at least two labeled nucleic acid probes, each having a different fluorescent label, said at least two labeled nucleic acid probes being a proximal probe and a distal probe specific to a gene.

### Example 14

The method of any of examples 11 through 13, said at least one labeled nucleic acid probe being specific for a target sequence.

### Example 15

The method of any of examples 11 through 13, said at least one labeled nucleic acid probe being specific for a target sequence, said target sequence being indicative of a disease state.

### Example 16

The method of any of examples 11 through 13, said at least one labeled nucleic acid probe being specific for a target sequence, said target sequence being indicative of a gene rearrangement.

### Example 17

The method of any of examples 11 through 13, said at least one labeled nucleic acid probe being specific for a target sequence, said target sequence being indicative of a chromosomal rearrangement.

### Example 18

The method of any of examples 1 through 17, further comprising determining a presence or absence of fluorescence on said bone marrow aspirate.

### Example 19

The method of any of examples 11 through 17, further comprising visualizing said at least one labeled nucleic acid probe.

### Example 20

A method for detecting a nucleic acid sequence in a previously frozen bone marrow aspirate (BMA) sample comprising: contacting said BMA sample with a first fixation solution comprising formalin; contacting said BMA sample with a second fixation solution; and contacting said BMA sample with a fluorescent labeled nucleic acid probe.

### Example 21

The method of example 20, said fluorescent labeled nucleic acid probe being configured to determine a phenotypic profile.

### Example 22

The method of any of examples 20 through 21, said fluorescent labeled nucleic acid probe being configured to detect a chromosomal rearrangement.

### Example 23

The method of example 22, said chromosomal rearrangement being diagnostic of acute myeloid leukemia (AML).

### Example 24

The method of any of examples 22 through 23, said chromosomal rearrangement being predictive of a therapeutic strategy for an individual from which said BMA sample was obtained.

### Example 25

An automated slide staining apparatus for assaying one or more tissue samples disposed on a slide, the apparatus comprising: a controller; a slide staining assembly, the slide staining assembly being configured to receive one or more of the slides; and at least one fluid dispensing robot configured by the controller to dispense a plurality of reagents to the one or more slides received in the slide staining assembly to treat the one or more tissue samples respectively, the at least one fluid dispensing robot being configured by the controller to dispense one or more reagents of the plurality of reagents in a predetermined sequence corresponding to a FISH protocol for the one or more slides received in the slide staining assembly to treat the one or more tissue samples disposed on each slide independently, the at least one fluid dispensing robot being further configured by the controller to dispense a first fixation solution comprising formalin and a second fixation solution comprising methanol and acetic acid, in a predetermined sequence corresponding to a FISH protocol for the one or more slides received in the slide staining assembly to treat the one or more tissue samples disposed on each slide independently, and the at least one fluid dispensing robot being further configured by the controller to perform the predetermined sequence corresponding to the FISH protocol.

### Example 26

The apparatus example 25, the slide staining assembly being configured by the controller to heat each slide received by the slide staining assembly independently to an incubation temperature.

### Example 27

The apparatus of any of examples 25 through 26, the slide staining assembly being configured by the controller to heat each slide received by the slide staining assembly independently to an incubation temperature during the staining protocol.

### Example 28

A method for an automated FISH assay of one or more tissue samples disposed on a slide, the method comprising dispensing one or more reagents to the one or more tissue samples using one or more automated fluid dispensers in a series of staining steps associated with a slide staining protocol.

### Example 29

The method of example 28, further comprising dispensing one or more reagents to the one or more tissue samples using one or more automated fluid dispensers in a series of sample preparation steps associated with a sample preparation protocol.

### Example 30

The method of any of examples 28 through 29, the one or more reagents comprising a nucleic acid probe configured to detect a nucleic acid sequence of interest.

### Example 31

The method of example 30, said nucleic acid sequence of interest being indicative of a chromosomal rearrangement.

### Example 32

The method of any of examples 29 through 31, the sample preparation protocol and the slide staining protocol being performed using the same instrument.

### IV. Conclusion

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following examples and is understood not to be limited to the details of structure and operation shown and described in the specification and drawings.

I/we claim:

1. A method of processing a bone marrow aspirate (BMA) sample for detection of a targeted nucleic acid sequence comprising
a first fixation step comprising contacting said BMA sample with a first fixation solution comprising formalin, and
a second fixation step comprising contacting said BMA sample with a second fixation solution comprising methanol and acetic acid.

2. The method of claim 1, said BMA being an unprocessed BMA sample.

3. The method of claim 1, said BMA being previously frozen.

4. The method of claim 1, said first fixation solution comprising from about 5% to about 20% formalin.

5. The method of claim 1, said first fixation solution comprising 10% formalin.

6. The method of claim 1, said second fixation solution comprising methanol and acetic acid at a ratio of about 3 to about 1.

7. The method of claim 1, said second fixation step being carried out following said first fixation step.

8. The method of claim 1, said second fixation step being followed by a drying step.

9. The method of claim 1, said BMA sample being applied to a first slide prior to said first fixation step, further comprising
placing a second slide at an angle to said first slide;
lowering said second slide to cover the BMA sample placed on said first slide; and
sliding said second slide across said first slide to create a BMA sample smear.

10. The method of claim 1, further comprising contacting said BMA sample with a proteolytic enzyme.

11. The method of claim 1, further comprising contacting said BMA sample with at least one labeled nucleic acid probe.

12. The method of claim 11, said at least one labeled nucleic acid probe being fluorescently labeled.

13. The method of claim 11, said at least one labeled nucleic acid probe comprising at least two labeled nucleic acid probes, each having a different fluorescent label, said at least two labeled nucleic acid probes being a proximal probe and a distal probe specific to a gene.

14. The method of claim 11, said at least one labeled nucleic acid probe being specific for a target sequence.

15. The method of claim 11, said at least one labeled nucleic acid probe being specific for a target sequence, said target sequence being indicative of a disease state.

16. The method of claim 11, said at least one labeled nucleic acid probe being specific for a target sequence, said target sequence being indicative of a gene rearrangement.

17. The method of claim 11, said at least one labeled nucleic acid probe being specific for a target sequence, said target sequence being indicative of a chromosomal rearrangement.

18. The method of claim 1, further comprising determining a presence or absence of fluorescence on said bone marrow aspirate.

19. The method of claim 11, further comprising visualizing said at least one labeled nucleic acid probe.

20. An automated slide staining apparatus for assaying one or more tissue samples disposed on a slide, the apparatus comprising:
(a) a controller;
(b) a slide staining assembly, the slide staining assembly being configured to receive one or more of the slides; and
(c) at least one fluid dispensing robot configured by the controller to dispense a plurality of reagents to the one or more slides received in the slide staining assembly to treat the one or more tissue samples respectively,
the at least one fluid dispensing robot being configured by the controller to dispense one or more reagents of the plurality of reagents in a predetermined sequence corresponding to a FISH protocol for the one or more slides received in the slide staining assembly to treat the one or more tissue samples disposed on each slide independently,
the at least one fluid dispensing robot being further configured by the controller to dispense a first fixation solution comprising formalin and a second fixation solution comprising methanol and acetic acid, in a predetermined sequence corresponding to a FISH staining protocol for the one or more slides received in the slide staining assembly to treat the one or more tissue samples disposed on each slide independently, and
the at least one fluid dispensing robot being further configured by the controller to perform the predetermined sequence corresponding to the FISH protocol.
